(19) United States
(12) Patent Application Publication
(10) Pub. No.: US 2024/0006020 A1
(43) Pub. Date: Jan. 4, 2024

(54) METHOD FOR PERFORMING IMPUTATION AND/OR ENRICHMENT OF GENETIC DATA IN AN OPTIMIZED MANNER

(71) Applicant: ALLELICA S.R.L., Rome (IT)

(72) Inventors: Paolo DI DOMENICO, Rome (IT); Giordano BOTTA', Rome (IT)

(21) Appl. No.: 18/252,792

(22) PCT Filed: Nov. 10, 2021
(86) PCT No.: PCT/IB2021/060403

§ 371 Date: May 12, 2023

(30) Foreign Application Priority Data
Nov. 13, 2020 (IT) 10 2020 000027188

(51) Int. Cl. G16B 20/20; G16B 40/20

(52) U.S. Cl. CPC G16B 20/20; G16B 40/20

(57) ABSTRACT

A method performs imputation and/or enrichment of genetic data by electronic computation. The method accesses partial information of the individual's genetic data set, available following detection by sequencing. The method partitions the genetic data set into a group of disjoint genetic data subsets so that the union of the subsets corresponds to the acquired genetic data set. The genetic data subsets have a same dimension based on quality criterion to be complied with by genetic data imputation/enrichment corresponding to genetic data contained. The subsets are processed in parallel, by applying to each genetic data subset, a genetic data imputation algorithm, to compare a partial genetic data set with the complete known genome of reference individuals. The method obtains enriched genetic data subsets, each being an enriched version of a respective genetic data subset. An enriched version of the individual's genetic data set is determined from the genetic data imputation/enrichment.

### FIELD OF APPLICATION

The present invention relates to a method for performing imputation and/or enrichment of genetic data in an optimized manner.

Therefore, the general technical field of the present invention is that of processing genetic data, performed by electronic computation, in support of a wide plurality of medical or clinical research applications, such as predictive and/or diagnostic prognoses.

More in particular, the present invention relates to a method for performing imputation and/or enrichment of genetic data by processing data in parallel.

### DESCRIPTION OF THE PRIOR ART

The use of genetic data has now established itself as indispensable information not only for the diagnosis of rare diseases, but also for the prevention of common and multi-factor diseases such as type-2 diabetes, cardio-vascular problems.

The information extracted from DNA is also of fundamental use for the prevention of oncological diseases, first and foremost breast, ovarian and prostate cancer.

This “genetic revolution” has led to the birth of different methods of extracting genetic data.

Whole sequencing (Whole Genome Sequencing—WGS) has been accompanied by less expensive techniques such as Micro Array, for example GSA by Illumina or AXIOM by Thermofisher), exome sequencing Whole Exome Sequencing—WES, and more recently WGS techniques carried out with low coverage (Low Coverage WES).

Unlike WGS, these new techniques do not perform a complete scan of the genetic patrimony of an individual (G), but select a subset of this information obtaining a reduced data set (S).

To perform some of the aforementioned predictive analyses, about 7 million genetic variants are also required, which are often not present in S.

In order to overcome this problem, data enrichment techniques are used, also referred to as “imputation” techniques, which allow, from the known subset S, obtaining a set as close as possible to G.

Imputation is a technique which compares S with the complete genetic data of a population of individuals for which G is known (referred to as “Reference Panel”) and through the use of different algorithms (Hidden Markov Model and Bayesian systems).

These techniques are computationally demanding and the present patent application presents an invention capable of optimizing this process.

### SUMMARY OF THE INVENTION

It is the object of the present invention to provide a method for performing imputation and/or enrichment of genetic data, by means of electronic computation, which allows solving, at least partially, the drawbacks claimed above with reference to the prior art, and responding to the aforementioned needs particularly felt in the technical field considered. Such an object is achieved by a method according to claim 1.

Further embodiments of such a method are defined in claims 2-9.

### BRIEF DESCRIPTION OF THE DRAWINGS

Further features and advantages of the method according to the invention will become apparent from the following description of preferred exemplary embodiments, given by way of non-limiting indication, with reference to the accompanying drawings, in which:

FIG. 1 shows a simplified block diagram of a system adapted to implement an embodiment of the method according to the invention.

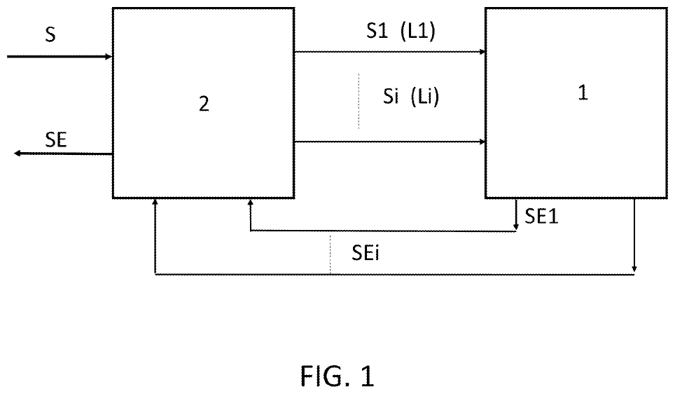

### DETAILED DESCRIPTION

A method for performing imputation and/or enrichment of genetic data, by electronic computation, is described.

Such a method comprises a step of accessing partial information of an individual's genetic patrimony, represented by a genetic data set S of the individual, available following a detection by means of a sequencing technique.

The method thus includes partitioning the aforesaid genetic data set S into a group of mutually disjoint genetic data subsets or chunks Si and such that the union of the aforesaid subsets or chunks Si corresponds to the acquired genetic data set S.

The aforesaid genetic data subsets Si have a same dimension Li, corresponding to the amount of genetic data contained.

Such a dimension Li is a pre-determined minimum dimension, based on a predetermined quality criterion to be complied with by the genetic data imputation and/or enrichment.

The method then comprises the step of processing the aforesaid subsets Si in parallel, by means of a first electronic processor 1 capable of performing parallel processing, by applying in parallel, to each of said genetic data subsets Si, at least one genetic data imputation algorithm, adapted to enrich the genetic information by comparing a partial genetic data set with the complete known genome G of one or more reference individuals.

The method then includes obtaining, as results of the aforesaid parallel processing step, a plurality of enriched genetic data subsets SEᵢ, in which each of such enriched subsets SEᵢ is an enriched version of a respective genetic data subset Si.

Finally, the method comprises the step of determining, as a result of the genetic data imputation and/or enrichment, an enriched version SE of the individual's genetic data set S, based on the aforesaid enriched subsets SEᵢ.

According to an embodiment of the method, the aforesaid first electronic processor capable of performing parallel processing is a Graphical Processing Unit (GPU).

In accordance with an embodiment of the method, the aforesaid steps of partitioning the genetic data set S and determining the enriched version SE of the genetic data set S are carried out by a second electronic processor 2, comprising for example conventional Control Processing Unit (CPU).

In such a case, the method further comprises the following steps.

After the step of partitioning, the method includes sending digital data corresponding to the subsets or chunks Si determined by the partition, from a main memory controlled by the second electronic processor to a memory of the first electronic processor.

After the step of obtaining a plurality of enriched subsets SEᵢ, the method includes sending digital data corresponding to the enriched subsets SEᵢ, from the memory of the first electronic processor to the main memory controlled by the second electronic processor.

According to an embodiment, the method employs genetic data imputation algorithms known per se. In fact, the present method can be advantageously performed on a wide plurality of known and commonly used imputation algorithms, since the application of the steps of the method, and in particular the execution of the processing parallelization, is not limited per se to specific algorithms.

According to an embodiment, the method performs parallel processing by GPU, for example, on a part of the imputation process which uses the BWT (Barrows-Wheeler Transform) algorithm, known per se in literature.

According to an embodiment of the method, the aforesaid step of determining the result of the genetic data imputation and/or enrichment comprises determining the enriched version SE of the genetic data set S of the individual as a union set of the aforesaid enriched subsets SEᵢ.

According to an implementation option, the generation of the aforementioned union set (i.e., a digital data set corresponding to the union of the digital data of all the enriched subsets obtained by parallel processing the chunks of genetic data) is carried out by the second processor electronic or CPU.

In accordance with an embodiment of the method, the aforesaid genetic data set S comprises a set of the individual's Single Nucleotide Polymorphisms (SNP). In this case, the genetic data subsets or chunks Si comprise respective subsets of the individual's Single Nucleotide Polymorphisms SNPi, and the aforesaid dimension of the subsets Si corresponds to the number of Single Nucleotide Polymorphisms SNPi contained.

According to an embodiment, the method comprises the further preliminary step of determining the dimension Li of the subsets Si as a minimum number of Single Nucleotide Polymorphisms which allow each subset or chunk Si to give rise to an enriched subset SEᵢ which complies with a predetermined quality criterion.

According to an implementation option, the aforesaid preliminary step of determining the dimension Li of the subsets Si comprises the following steps:
defining, based on known data, a genetic reference data (TrueGenotype);
eliminating from the set of SNPs of the genetic data subset to be evaluated S the SNPs which are not present in the general reference data (TrueGenotype), thus obtaining a modified set Sm;
partitioning the aforesaid modified set Sm into chunks having a test dimension Lₜₑₛₜ;
performing a test imputation determination, by an imputation algorithm selected on the aforesaid modified set;
calculating an imputation quality parameter on the test determination results;
varying the test dimension (Lₜₑₛₜ) according to a predetermined rule;
iterating said steps of performing a test determination, calculating an imputation quality parameter and varying the test dimension (Lₜₑₛₜ) up to maximizing the imputation quality parameter;
determining, as the dimension Li of the subsets Si, the resulting test dimension at the end of the iteration.

In accordance with an implementation option of the method, the aforesaid step of varying the test dimension Lₜₑₛₜ according to a predetermined rule comprises considering dimensions increased and decreased by an amount equal to Lₜₑₛₜ/2 as the next test dimensions.

In this case, the calculation step comprises calculating an imputation quality parameter on the results of the two further test dimensions, equal to Lₜₑₛₜ+Lₜₑₛₜ/2 and Lₜₑₛₜ−Lₜₑₛₜ/2, respectively; if the imputation quality in the two cases is similar, (i.e., showing a deviation below a certain threshold), the smaller chunk is chosen; if the deviation between the imputation qualities in the two cases is greater than said certain threshold, the larger chunk is chosen.

According to a particular implementation option of the method, the step of calculating an imputation quality parameter is carried out by means of a “NON-REF Concordance” technique, which consists in finding the percentage of correctly imputed SNPs among all the SNPs having at least one allele with a variant in ALT.

According to an implementation option of the method, the step of calculating an imputation quality parameter is carried out based on a comparison of the imputed data with the reference genetic data (TrueGenotype).

As can be seen, the objects of the present invention as previously indicated are fully achieved by the method described above by virtue of the features disclosed above in detail.

In fact, the solution disclosed above allows implementing data enrichment techniques, or “imputation” techniques, which allow, from the known subset S, obtaining a set as close as possible to G, faster and more effectively than the conventional techniques used.

Such a technical advantage is achieved not only by virtue of the application of parallel computing, and not only by virtue of the use of a processor specialized in parallel processing (for example, GPU), but also by virtue of the specific methods, disclosed above, through which genetic data are treated, partitioned, and processed.

In order to meet contingent needs, those skilled in the art may make changes and adaptations to the embodiments of the method described above or can replace elements with others which are functionally equivalent without departing from the scope of the following claims. All the features described above as belonging to a possible embodiment may be implemented irrespective of the other embodiments described.

1. A method for performing imputation and/or enrichment of genetic data, by electronic computation, comprising:
accessing partial information of an individual's genetic patrimony, represented by a genetic data set of the individual, available following a detection by a sequencing technique;
partitioning said genetic data set into a group of genetic data subsets or chunks, mutually disjoint, so that a union of said genetic data subsets corresponds to the genetic data set,
wherein said genetic data subsets have a same dimension, corresponding to an amount of genetic data contained,
wherein said dimension is a pre-determined minimum dimension, based on a predetermined quality criterion to be complied with by the genetic data imputation and/or enrichment;
processing said subsets in parallel, by a first electronic processor capable of performing parallel processing, by applying in parallel, to each of said genetic data subsets, at least one genetic data imputation algorithm, adapted to enrich the genetic information by comparing a partial genetic data set with the complete known genome of one or more reference individuals;
obtaining, as results of said parallel processing step, a plurality of enriched genetic data subsets, each enriched subset being an enriched version of a respective genetic data subset;
determining, as a result of the genetic data imputation and/or enrichment, an enriched version of said genetic data set of the individual, based on said enriched subsets.

2. The method according to claim 1, wherein said first electronic processor capable of performing parallel processing is a Graphical Processing Unit.

3. A method according to claim 2, wherein said steps of partitioning the genetic data set and determining the enriched version of the genetic data set are carried out by a second electronic processor, said second electronic processor being a conventional Control Processing Unit, and wherein the method comprises the further steps of:
after the step of partitioning, sending digital data corresponding to the subsets or chunks determined by the partition, from a main memory controlled by the second electronic processor to a memory of the first electronic processor;
after the step of obtaining a plurality of enriched subsets, sending digital data corresponding to the enriched subsets, from the memory of the first electronic processor to the main memory controlled by the second electronic processor.

4. A method according to claim 1, wherein said step of determining the result of the genetic data imputation and/or enrichment comprises determining the enriched version of said genetic data set of the individual as the union set of said enriched subsets (SEᵢ).

5. A method according to claim 1, wherein said genetic data set comprises a set of Single Nucleotide Polymorphisms of the individual, and wherein said subsets or chunks comprise respective subsets of the individual's Single Nucleotide Polymorphisms,
and wherein said dimension of the subsets corresponds to the number of Single Nucleotide Polymorphisms contained.

6. A method according to claim 1, comprising the further preliminary step of determining the dimension of the subsets as a minimum number of Single Nucleotide Polymorphisms N which allow each subset or “chunk” to give rise to an enriched subset which complies with a predetermined quality criterion.

7. A method according to claim 5, wherein said preliminary step of determining the dimension of the subsets comprises the following steps:
defining, based on known data, a genetic reference data;
eliminating from the set of single nucleotide polymorphisms of the genetic data subset to be evaluated the single nucleotide polymorphisms which are not present in the general reference data, thus obtaining a modified set;
partitioning said modified set into chunks having a test dimension;
performing a test imputation determination, by an imputation algorithm selected on said modified set;
calculating an imputation quality parameter on the test determination results;
varying the test dimension according to a predetermined rule;
iterating said steps of performing a test determination, calculating an imputation quality parameter, and varying the test dimension up to maximizing the imputation quality parameter;
determining, as the dimension of the subsets, the resulting test dimension at the end of the iteration.

8. A method according to claim 7, wherein said step of varying the test dimension according to a predetermined rule comprises considering dimensions increased and decreased by an amount equal to one half the test dimension as the next test dimensions,
wherein the step of calculating comprises calculating an imputation quality parameter on the results of the two further test dimensions equal to the test dimension plus one half the test dimension, and the test dimension minus one half the test dimension; if the imputation quality in the two further test dimensions is similar, having a deviation below a certain threshold, the smaller chunk is chosen; if the deviation between the imputation qualities in the two cases is greater than a certain threshold, the larger chunk is chosen.

9. A method according to claim 7, wherein the step of calculating an imputation quality parameter is carried out by a “NON-REF Concordance” technique, which includes finding a percentage of correctly imputed single nucleotide polymorphisms among all the single nucleotide polymorphisms having at least one allele with a variant in ALT,
or wherein the step of calculating an imputation quality parameter is carried out based on a comparison of the imputed data with the reference genetic data.
